(19) United States
(12) Patent Application Publication
(10) Pub. No.: US 2018/0064330 A1
(43) Pub. Date: Mar. 8, 2018

(54) VISION ASSISTANCE SYSTEM

(71) Applicants: Leonard MARKUS, Sanctuary Cove (AU); Michael Henry KENDALL, Sanctuary Cove (AU)

(72) Inventors: Leonard MARKUS, Sanctuary Cove (AU); Michael Henry KENDALL, Sanctuary Cove (AU)

(21) Appl. No.: 15/560,261

(22) PCT Filed: Mar. 23, 2016
(86) PCT No.: PCT/AU2016/000100

§ 371 Date: Sep. 21, 2017

(30) Foreign Application Priority Data
Mar. 23, 2015 (AU) 2015901034
Jun. 2, 2015 (AU) 2015100739

(51) Int. Cl. A61B 3/00; A61B 3/032; G06F 3/0484; G06F 3/0488; G09B 21/00

(52) U.S. Cl. CPC A61B 3/0075; A61B 3/032; G09B 21/008; G06F 3/04883; G06F 3/04847

(57) ABSTRACT

A vision assistance method implemented on a digital display device provides a test image for display to a user on a display of the graphical user interface of the device, and an adjustment interface, configured to be displayed adjacent to the test image on the display and enables the user to input visual adjustment settings to adjust the test image. The test image is then automatically adjusted by a processor of the device by applying the adjustment settings. A profile of the user is generated including the desired visual adjustment settings iteratively selected by the user. An input image is subsequently received and the processor then automatically adjusts the input image based upon visual adjustment data corresponding to the selected desired visual adjustment settings. The adjusted input image is then displayed on the display and to the user.

### TECHNICAL FIELD

The present invention relates to vision assistance. In particular, although not exclusively, the present invention relates to adaptation of a digital display of a user device to compensate for a vision impairment of a user.

### BACKGROUND ART

Over the years, people have become more and more reliant on good eye sight. In particular, daily tasks generally require the ability to read small text in books, on digital displays (e.g. computer screens), on far away street signs and the like. As a result, eye glasses have, over time, become very important in correcting vision problems or impairments, such as farsightedness and shortsightedness.

A problem with glasses is that they are generally bulky and uncomfortable, particularly when used for extended periods. This is especially evident when considering that much of modem daily life is spent viewing devices with digital displays.

Alternatives to glasses exist, including contact lenses. However, contact lenses have problems of their own, including causing irritation to the eye and dry eyes. Furthermore, some people find contact lenses uncomfortable, particularly when used for long periods.

Modem day life often involves digital display devices, including mobile phones, from early in the morning until late at night. In fact, it is common for people to view smartphones immediately prior to going to bed, and when waking up in the morning. As a result, eye glasses and contact lenses are not particularly suited to the prolonged use of digital display devices required in modern day life.

Certain system exist that aim to assist users with eye problems in reading text on digital display devices. Such systems typically enlarge and increase the contrast of the text. However, such systems are generally not suited to portable digital display devices, such as smartphones, as only a very small amount of text can be displayed on the screen at a time. Furthermore, such systems generally remove important aesthetic details associated with the text, including colour,

background and the like,

Accordingly, there s a need for proved vision assistance system.

### SUMMARY OF INVENTION

The present invention is directed to vision assistance systems and methods, which may at least partially overcome at least one of the abovementioned disadvantages or provide the consumer with a useful or commercial choice.

With the foregoing in view, the present invention in one form, resides broadly in a vision assistance method implemented on a digital display device, the method comprising:

receiving an input image on a display of the device;

adjusting, by a processor, the input image based upon visual adjustment data of a user;

displaying, on the display and to the user, the adjusted image.

Advantageously, certain embodiments of the invention enable users with vision problems to view images on the display without the need for any external vision correction devices, such as eye glasses, as the input image is instead adjusted based upon visual adjustment data of the user.

The visual adjustment data may include data to compensate or correct for a refractive error of the eyes of the user, wherein the adjusted image at least partly compensates for the refractive error, and/or data to compensate or correct for colour blindness, and/or data to compensate for the location and movement of the face (and especially the eyes) of the user, and which is derived from sensor(s) in the digital display device.

Accordingly, the present invention provides a vision assistance method implemented on a digital display device, the method comprising:
(a) providing, to a user, a graphical user interface of the device, the graphical user interface including:
   (i) a test image for display to the user on a display of the graphical user interface, and
   (ii) an adjustment interface configured to be displayed adjacent to the test image on the display and for enabling the user to input visual adjustment settings to adjust the test image;
(b) automatically adjusting, by a processor of the device, the test image on the display by applying to the test image the visual adjustment settings input by the user who iteratively selects desired visual adjustment settings using the test image and the adjustment interface;
(c) subsequently receiving an input image;
(d) automatically adjusting, by the processor, the input image based upon visual adjustment data corresponding to the selected desired visual adjustment settings; and
(e) displaying, on the display and to the user, the adjusted input image.

Advantageously, certain embodiments of the invention enable users to determine visual adjustment data by adjusting settings of an input image, and viewing the result of each of the settings, until the input image is of an acceptable standard to the user.

The visual adjustment data may include colour compensation data, wherein the input image is adjusted to at least partly alleviate colour blindness of the user by adjusting one or more colours of he input image using the colour compensation data.

As such, the invention enables a colour blind person to differentiate between different colours in an image in such a way that would not otherwise have been possible.

The method may further comprise:
(f) saving the visual adjustment data of the user in database including saved visual adjustment data of a plurality of users;
(g) subsequently retrieving the saved visual adjustment; data of a second user from the database;
(h) receiving a further input image;
(i) automatically adjusting, by the processor, the further input image based upon the saved visual adjustment data of the second user; and
(j) displaying, on the display and to the second user, the adjusted further input image.

Advantageously, certain embodiments of the invention enable several users with different vision problems to view images on the display, without the need for any external vision correction devices, such as eye glasses, as the input image is instead adjusted based upon the respective users visual adjustment data.

The visual adjustment data of the user may comprise or be contained in, a profile of the user. The visual adjustment data may include data to compensate for refractive error of the eyes of the user or for colour blindness of the user. The visual adjustment data may also include eye related data, such as pupillary distance.

The method may comprise generating the visual adjustment data based upon input from the user. The visual adjustment data may be generated by providing a plurality of images to the user, wherein the images are generated according to different visual adjustment data. The images may be generated based upon input from the user.

The method may further comprise generating the graphical user interface, for determining the visual adjustment data of the user. The graphical user interface may include: at least one adjustment interface, for enabling the user to input visual adjustment settings; and an image, on which the input visual adjustment settings are automatically applied. The image may be a test image, which is automatically adjusted or modified based upon the input visual adjustment settings.

The graphical user interface may be generated upon determining that visual adjustment settings for the user are not available.

The method may comprise retrieving saved visual adjustment data for the user from a database including saved visual adjustment data of a plurality of users.

The input image may comprise an image from a plurality of images of a video sequence, wherein each image of the plurality of images of the video sequence is adjusted and displayed according to the visual adjustment data.

The input image may be adjusted by applying an image filter to the input image. The image filter may comprise a deconvolution filter.

The display may include a lens for selectively adjusting pixels of the display. In such a case, the test image or the input image may be adjusted by moving pixels in the test image or the input image such that a first set of the pixels is adjusted by the lens in a first manner, and a second set of the pixels is adjusted by the lens in a second manner.

The lens may be configured to direct light from the different sets of pixels in different directions. The lens may include at least three directional components, for directing image data in at least three different directions. The at least three directional components may be repeated across the lens.

The visual adjustment data may also include dynamic visual adjustment data to compensate for the location and movement of the face (and especially the eyes) of the user, and which is derived from sensor(s) in the digital display device.

The display may be a display of a smartphone.

In another form, the present invention resides in a vision assistance system, the system comprising:

a data interface for receiving an input image;

a processor, coupled to the data interface, for adjusting the input image based upon visual adjustment data of a user; and

a display for displaying the adjusted image to the user.

The display may include a lens for directing light from the display in different directions.

In yet another form, the present invention resides in a personal computing device comprising:

a graphical user interface for receiving an input image;

a processor, coupled to the graphical user interface, for adjusting the input image based upon visual adjustment data of a user; and

a display for displaying the adjusted image to the user.

In yet another form, the present invention resides in a lens for attaching a display, the lens configured to adjust an output of the display to compensate for a vision problem of a user. The lens may include an adhesive for attaching the lens to the display. The lens may be releasably attachable to the display. The lens may also protect the display from scratches.

Any of the features described herein can be combined in any combination with any one or more of the other features described herein within the scope of the invention.

### BRIEF DESCRIPTION OF DRAWINGS

Various embodiments of the invention will be described with reference to the following drawings, in which:

FIG. 1 illustrates a vision assistance system according to an embodiment of the present invention;

FIG. 2a illustrates a screenshot of a configuration screen, according to an embodiment of the present invention;

FIG. 2b illustrates a further screenshot of the configuration screen of FIG. 2a, after it has been adjusted by the user,

FIG. 3 illustrates a vision assistance method according to an embodiment of the present invention;

FIG. 4 illustrates a vision adjustment configuration method according to an embodiment of the present invention; and

FIG. 5 illustrates a cross section of a display screen according to an embodiment of the present invention.

### DESCRIPTION OF EMBODIMENTS

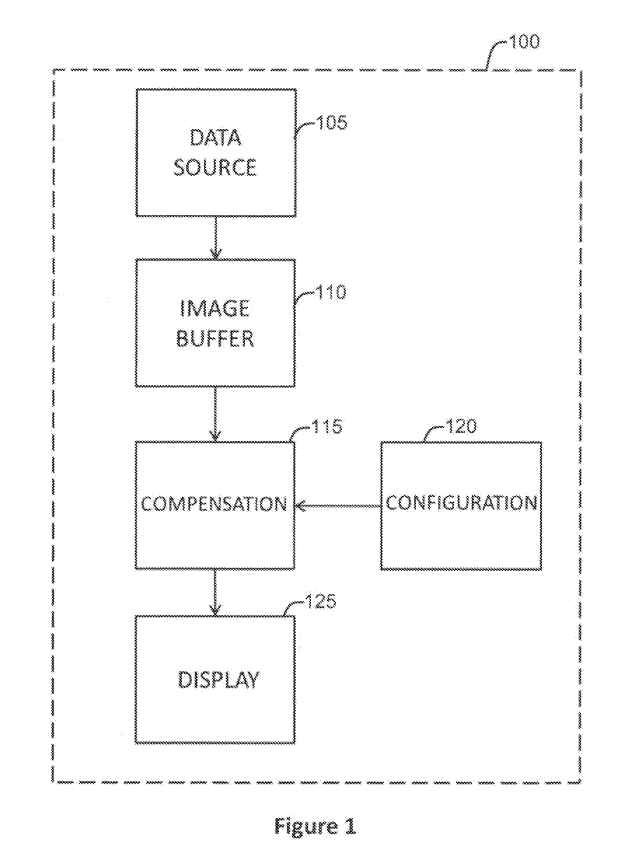

FIG. 1 illustrates a vision assistance system 100 according to an embodiment of the present invention. The vision as system 100 enables a person with vision problems to view a digital display on a user device without needing to wear corrective lenses.

The vision assistance system 100 includes a data source 105 for providing display data. The data source may comprise image data associated with a digital book or magazine, a website, an app (e.g. email or word processing), a video, photographs, or any other image data that may be displayed. The image data is then rendered onto an image buffer 110.

The image buffer 110 may comprise a portion of mere memory associated with the display of image data, such as dedicated graphics memory. The image buffer 110 may be timed such that data is written to the image buffer 110 at particular times, such as 30 times per second for video data.

A compensation module 115, which is coupled to the image buffer 110, compensates for a vision problem associated with the person. In particular, the image data of the image buffer is modified to suit the vision problem of the user.

The image data is modified using an it filter. The image filter may operate in the pixel domain, the frequency domain, the wavelet domain or a combination thereof.

Examples of filters include deconvolution filters (such a Wiener deconvolution filter), however any suitable filter may be used.

As described in further detail below, the image data may be modified according to a lens of the display. In particular, pixel data may be moved between pixels to provide different characteristics to the pixel data based upon the lens configuration.

The term “compensation” does not imply that the vision problem is entirely remedied (or compensated for) by the compensation module 115, but instead that adjustments are made to improve a perceived quality of the image when viewed by the user.

A configuration module 120 is in communication with the compensation module 115 to enable the compensation module 115 to be configured to a particular user. As described in further detail below, the configuration module 120 may provide test images to the person, together with adjustment means, to adjust the processing of the image data to suit the user. Alternatively or additionally, the configuration module 120 may receive input from the user in relation to a vision problem, prescription details or the like.

Finally, a display 125 is in communication with the compensation module 115 for displaying an image that has been compensated (or adjusted) to suit the user. The display may comprise a liquid-crystal display (LCD), a light-emitting diode (LED) display, an organic LED (OLED) display, or any other suitable display.

The vision assistance system 100 may comprise part of a digital display device, such as a smartphone, a television, a personal computer or the like. Alternatively, the vision assistance system 100 may be formed of a plurality of distinct devices, such as a user device and a server. In such a case, the compensation module 115 may configure the image data to the particular user remotely of the display device.

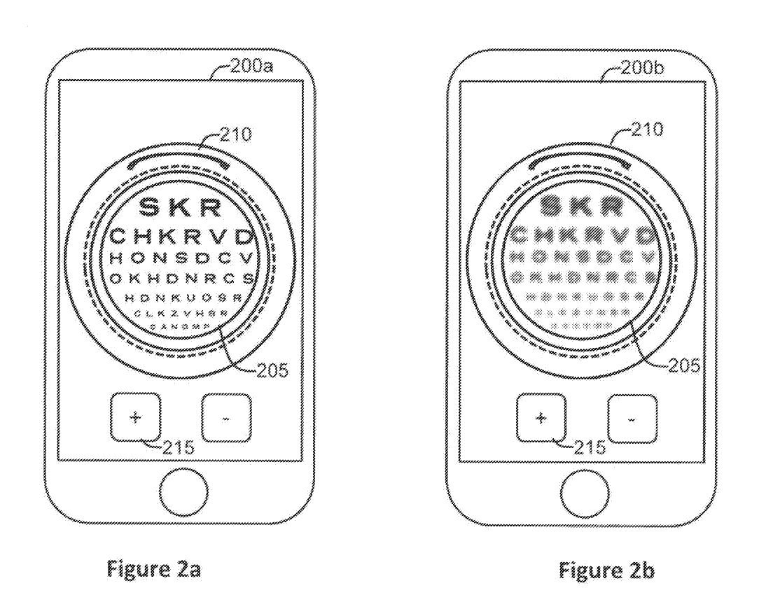

FIG. 2a illustrates a tree hot 200a of a configuration screen according to an embodiment of the present invention. The configuration screen may be similar or identical to a configuration screen of the configuration module 120 of FIG. 1. The configuration screen is illustrated with reference to a smartphone, however the skilled addressee will readily appreciate that the configuration screen may be easily adapted to suit a television, or any other suitable device.

The configuration screen includes a test image 205, a focus dial 210 and a plurality of adjustment buttons 215. The test image 205 comprises a plurality of characters of varying size and which are readily identifiable by the user as being blurry or sharp.

Upon rotation of the focus dial 210, the test image 205 is adjusted. The adjustment may correspond to, or be related to, a focus of the test image 205 in a similar manner to a focus arrangement of a camera or of a telescope. Similarly, when the adjustment buttons 215 are pushed, the test image 205 is also adjusted. As a result, the focus dial 210 may be used to compensate for refrective error of the eyes.

In the case of a smartphone having a touch screen, the focus dial 210 may be rotated using gesture input of the touch screen (e.g. rotating fingers on the screen).

The focus dial 210 and the adjustment buttons 215 may adjust separate aspects of the test image 205. Furthermore, further adjustment buttons 215 may be provided to enable adjustment of more than two aspects of the test image.

In use, the user will typically initially view the configuration screen with eye glasses (or other corrective lenses). However, this may not be required if the user is able to see the focus dial 210 and the adjustment buttons 215 sufficiently well without glasses.

The user will then take off their glasses (if needed) to rotate the focus dial 210 and/or push the adjustment buttons 215. The user will evaluate whether the initial adjustment caused an improvement in test image quality (e.g. sharpness), and may then rotate the focus dial 210 and/or push the adjustment buttons 215, either further, or back to a baseline setting if the initial adjustment caused a decrease in perceived test image quality.

The user may iteratively switch between the focus dial 210 and the adjustment buttons 215 when making adjustments to the test image. As a result, the user may adjust the quality of the test image 205 by considering one or more variables at a time.

FIG. 2b illustrates a further screenshot 200b of the configuration screen after it has been adjusted by the user. As illustrated, the adjusted test image 205 is blurry to a typical user, but compensates for sight problems of the particular user, and is thus sharp (or at least improved) to the particular user when compared with the unadjusted test image 205 shown in FIG. 2a.

According to certain embodiments (not illustrated), the test image 205 and the focus dial 210 and the adjustment buttons 215 are all adjusted simultaneously. As a result, the focus dial 210 and the adjustment buttons 215 may be clear to the user when adjusted, which may reduce the need to switch between viewing the display with and without glasses.

According to alternative embodiments, the test image 205 may comprise an image of the data to be displayed, e.g. an app, video data or the like. As a result, the user may choose and adjust settings depending on the data being used. In such a case, dark movie images and high contrast text, for example, may have different settings based upon user preference.

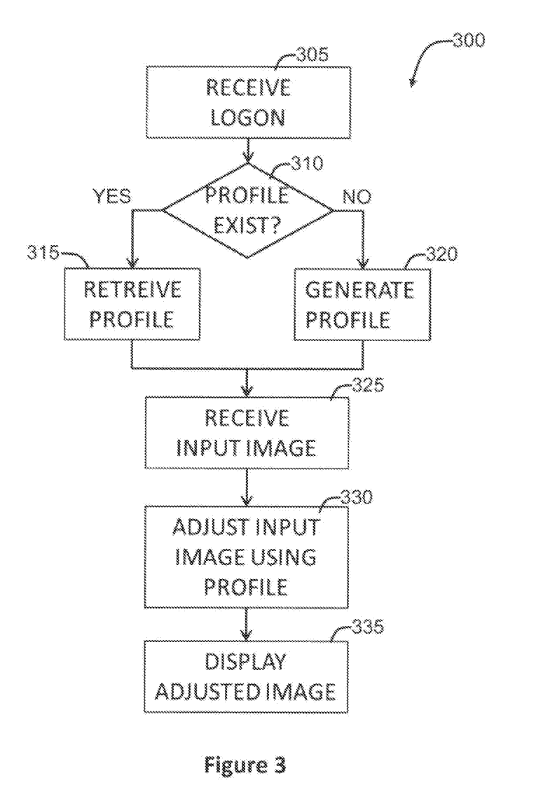

FIG. 3 illustrates a vision assistance method 300 according to an embodiment of the present invention.

At step 305, confirmation of a user logging on to the system is received. This may be through the selection of a user profile by the user, by entering a username and password, or by any other suitable means.

At step 310, it is determined if a profile, including visual adjustment settings, exists for the user. If yes, the profile is retrieved at step 315. The profile may be stored on a central server, and as a result, the profile may be shared across devices. Alternatively, the profile may be stored on the device.

If there is no profile available for the user, a profile is generated at step 320. The profile may be generated by adjusting the test image 205, in the manner as described earlier, using a configuration screen, as illustrated in FIG. 2a and FIG. 2b above. The visual adjustment settings contained in the profile may be used by that user for automatically adjusting a subsequently received input image. The adjustment of the input image is based upon visual adjustment data corresponding to the visual adjustment settings which were iteratively selected by the user when generating the profile.

At step 325, an input image is received. The input image is generally unmodified, and may, for example, be an image of a video sequence, a screen of an app, or any other image.

At step 330, the input image is adjusted according to the profile. In particular, the input image is adjusted according to the visual adjustment data such that the image can be viewed by the user without corrective eye glasses.

Also, the input image may be adjusted to compensate for the location and movement of the face (and especially the eyes) of the user. This dynamic visual adjustment data is derived from one or more sensors in the digital display device which enable automatic refocusing of the image on the display.

At step 335, the adjusted input image is displayed on a display and to the user.

In the case of video, or other dynamic image data, steps 325 to 335 may be repeated for each frame of the video or image data.

According to certain embodiments, the method enables storage of profiles for a plurality of users. As such, the user profile can be selected when logging in and automatically used to adjust images in a manner that is specific to that user.

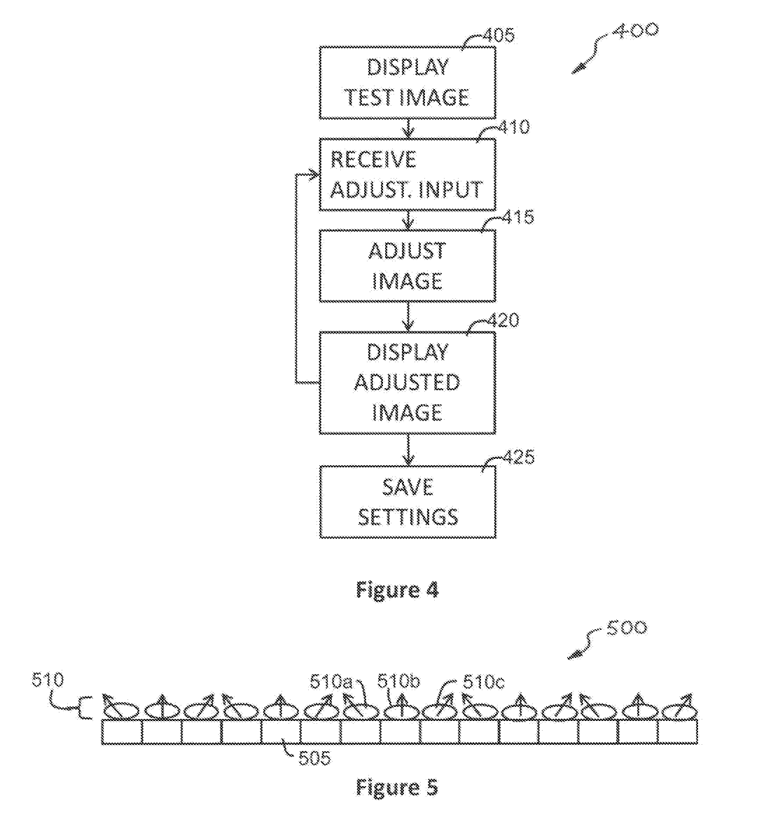

FIG. 4 illustrates a vision adjustment configuration method 400 according to an embodiment of the present invention. The adjustment configuration method 400 may be used to generate a profile as defined in step 320 of FIG. 3.

At step 405, a test image is displayed to the user. The test image may comprise a high frequency pattern, specifically designed to detect blurriness. Alternatively, the test image may comprise an image of the data that is to be adjusted on the device.

At step 410, adjustment input is received from the user. The adjustment input may comprise absolute input (e.g. a corrective factor), or a relative input (e.g. relative to a previous input). For example, the adjustment input may comprise input from the focus dial 210 (e.g. rotation input) and/or adjustment buttons 215 (e.g. push input) of FIGS. 2a and 2b.

At step 415, the image is adjusted based upon the received adjustment input. As previously mentioned, adjustment of the image may comprise compensating for a vision problem of a user, such as nearsightedness or farsightedness, or colour blindness.

At step 420, the adjusted image is displayed to the user. At this point, the user may determine whether the adjusted image is better than the test image. In such a case, the user may further adjust the associated setting, or if the image is of worse quality, the user may reverse the associated setting change by providing further adjustment input at step 410.

Steps 410, 415 and 420 are thus repeated to iteratively select desired visual adjustment settings until the user is satisfied with the adjusted image, or otherwise chooses to no longer refine the settings. At such a point, the settings, which may comprise, or be contained in, a user profile, are saved at step 425 and thereby constitute visual adjustment data that may subsequently be used to automatically adjust an input image.

FIG. 5 illustrates a cross section of a display screen 500 according to an embodiment of the present invention. The display screen 500 may be used together with any of the above methods and systems to assist in adjusting a test image and/or an input image to a user.

The display screen 500 includes a plurality of pixels 505, which are arranged in a two dimensional array (not illustrated). The display screen is generally rectangular, however any suitable shape may be used.

The display screen further includes a lens 510 adjacent the pixels 505. The lens 510 is configured to selectively adjust the pixels, in this case by directing light from the pixels 505 in different directions.

In particular, the lens includes a first directional component 510a, a second directional component 510b, and a third directional component 510c.

The components 510a, 510b, 510c are configured to direct the light from the pixels to the left (510a), vertically (510b) and to the right (510c), respectively.

The directional components 510a, 510b, 510c are repeated after every third pixel along the screen to enable a test image or an input image to be adjusted by moving the pixels rare more than two spaces to the side, while changing a directionality of the pixel. As a result, directionality can be provided without significantly distorting the image.

Three directional components (510a, 510b, 510c) are illustrated for the sake of simplicity, and the skilled addressee will readily appreciate that more than three directional components may be used. For example, four, five, six, seven, eight, nine, ten or more than ten directional components may be used.

According to alternative embodiments, the directional components (510a, 510b, 510c) are not evenly distributed across the screen. For example, outer pixels of the screen (e.g. pixels near the edge) may be given more directionality than central pixels.

The skilled addressee will readily appreciate that the lens 510 may be used together with any suitable signal processing method disclosed above.

In certain embodiments, the lens 510 is configured to adjust an output of the pixels 505 to compensate for a vision problem of a user. The lens 510 may include an adhesive, for attaching the lens 510 to a pre-manufactured display that includes the pixels 505. The lens may be releasably attachable to the pre-manufactured display. The lens may also protect the display from scratches.

According to certain embodiments, the display screen 500 comprises an auto stereoscopic display screen.

In alternative embodiments, the user may enter prescription details as a baseline for configuration. For example, in the configuration screen of FIG. 2a and FIG. 2b, the test image 205 may be initially displayed based upon the prescription details, and refined from there.

According to certain embodiments, a device is configured in a settings component of the device, upon which all apps, videos, images and the like are adjusted according to the settings.

The profile/adjustment input may relate directly or indirectly to eye related data, such as refractive error data, pupillary distance and the like.

According to certain embodiments, the systems and methods of the present invention can be used to compensate for colour blindness.

In certain types of colour blindness, users have difficulty distinguishing between red and green, and in other types, users have difficulty distinguishing between blue and yellow. Depending on the type of colour blindness, the systems and methods may adjust input images according to colour compensation data to at least partly alleviate the colour blindness of the user.

As an illustrative example, the colour compensation data of a person who has difficulty distinguishing between red and green may include a colour transform that transforms one or both of red and green of the input images to colours that are more easily differentiable by that person. As such, the colour compensation data may allow the user to differentiate between colours that were previously difficult to differentiate, rather than ‘reversing’ the colour blindness.

In another example, a user with mild colour blindness, e.g. a user who can differentiate between red and green, but with more difficulty than a non-colour blind user, may choose to enhance the red and green of the input images to assist in differentiation of same, rather than changing the colours as previously described.

According, to certain embodiments, a colour blind user is able to select the colour compensation data, or level of compensation applied, according to personal preferences. For example, a user with mild colour blindness may with to reduce colour compensation levels to avoid artificial looking colours, whereas another user may require high compensation levels to even be able to distinguish between colours.

In the present specification and claims the word ‘comprising’ and its derivatives including ‘comprises’ and ‘comprise’ have an inclusive meaning so as to include each of the stated integers and to not exclude one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations that would be readily understood by the skilled addressee.

1. A vision assistance method implemented on a digital display device, the method comprising:
(a) providing, to a user, a graphical user interface of said digital display device, the graphical user interface including:
(i) a test image for display to the user on a display of the graphical user interface; and
(ii) an adjustment interface configured to be displayed adjacent to the test image on the display and for enabling the user to input visual adjustment settings to adjust the test image;
(b) automatically adjusting, by means of a processor of said digital display device, the test image on the display by applying to the test image the visual adjustment settings input by the user who iteratively selects desired visual adjustment settings using the test image and the adjustment interface;
(c) subsequently receiving an input image;
(d) automatically adjusting, by means of said processor, the input image based upon visual adjustment data corresponding to the selected desired visual adjustment settings; and
(e) displaying, on the display and to the user, the adjusted input image.

2. The method of claim 1, wherein the adjustment interface enables the user to input visual adjustment settings relative to previously input visual adjustment settings.

3. The method of claim 1, wherein the adjustment interface comprises a focus dial and/or adjustment buttons.

4. The method of claim 1, wherein the visual adjustment data includes colour compensation data, and wherein the input image is adjusted to at least partly alleviate colour blindness of the user by adjusting one or more colours of the input image using the colour compensation data.

5. The method of claim 1, further comprising the steps of:
(f) saving the visual adjustment data of the user in a database including saved visual adjustment data of a plurality of users;
(g) subsequently retrieving the saved visual adjustment data of a second user from the database;
(h) receiving a further input image;
(i) automatically adjusting, by means of said processor, the further input image based upon the saved second visual adjustment data of the second user; and
(j) displaying, on the display and to the second user, the adjusted further input image.

6. The method of claim 1, wherein the visual adjustment data includes data to compensate for refractive error of the eyes of the user.

7. The method of claim 1, wherein the visual adjustment data is generated by providing a plurality of images to the user, wherein said plurality of images is generated from input from the user.

8. The method of claim 1, further comprising the step of generating the graphical user interface upon determining that visual adjustment data for the user is not available, the graphical user interface being generated by retrieving saved visual adjustment data for the user from a database including saved visual adjustment data of a plurality of users.

9. The method of claim 1, wherein the input image comprises an image from a plurality of images of a video sequence, further wherein each image of the plurality of images of the video sequence is adjusted and displayed according to the visual adjustment data.

10. The method of claim 1, wherein the input image is adjusted by applying an image filter to the input image.

11. The method of claim 10, wherein the image filter comprises a deconvolution filter.

12. The method of claim 1, wherein said display includes a lens for selectively adjusting pixels of the display, further wherein the test image or the input image is adjusted by moving pixels in the test image or the input image such that a first set of the pixels is adjusted by the lens in a first manner, and a second set of the pixels is adjusted by the lens in a second manner.

13. The method of claim 12, wherein the lens is configured to direct light from the first set of the pixels in a first direction and to direct light from the second set of 1 the pixels in a second direction which is different from said first direction.

14. The method of claim 13, wherein there is a third set of the pixels, and wherein the lens is further configured to direct light from the third set of the pixels in a third direction which is different to the first and second directions.

15. The method of claim 1, wherein the digital display device comprises one or more sensors configured to derive visual adjustment data to compensate for the location and movement of the face of the user.

16. The method of claim 1, wherein said display is a smartphone display.

17. A vision assistance system comprising:
a data interface for receiving an input image;
a processor, coupled to said data interface, for adjusting the input image based upon visual adjustment data of a user; and
a display for displaying the adjusted image to the user.

18. The vision assistance system of claim 17, wherein said system further comprises a lens for directing light from the display in different directions.

19. A personal computing device comprising:
a graphical user interface for receiving an input image;
a processor, coupled to said graphical user interface, for adjusting the input image based upon visual adjustment data of a user; and
a display for displaying the adjusted image to the user.
